(19) United States
(12) Patent Application Publication
(10) Pub. No.: US 2020/0038574 A1
(43) Pub. Date: Feb. 6, 2020

(54) APPARATUS FOR THE EXTRACORPOREAL TREATMENT OF BLOOD

(71) Applicant: IN10SIVECARE S.R.L., Salerno (IT)

(72) Inventor: Arturo RAMPOLLA, Palermo (IT)

(21) Appl. No.: 16/603,269

(22) PCT Filed: Apr. 7, 2018
(86) PCT No.: PCT/IB2018/052431

§ 371 Date: Oct. 7, 2019

(30) Foreign Application Priority Data
Apr. 7, 2017 (IT) 102017000038389

(51) Int. Cl. A61M 1/16; A61M 39/10; A61M 1/34; A61M 1/36

(52) U.S. Cl. CPC A61M 1/1698; A61M 1/1645; A61M 2205/3334; A61M 1/3413; A61M 1/3659; A61M 39/105

(57) ABSTRACT

An apparatus for the extracorporeal treatment of blood with veno-venous access, of the type comprising a circuit defined by a main pump (2) and by one or more conduits through which the blood to be treated passes at a given flow value (V1), said circuit being provided with an oxygenator (4), which performs a treatment on the blood at a first flow value, and of a hemofilter (7), which performs a treatment on the blood at a second flow value, lower than said first flow value. The apparatus is characterized in that: said oxygenator (4) is arranged and acting on a first portion (21) of the blood circuit and that the hemofilter (7) is arranged and acting on a second portion (22) of the blood circuit arranged parallel to the first portion (21); said second portion (22) is connected to the first portion.

The present invention relates to an apparatus for the extracorporeal treatment of blood by veno-venous access.

More precisely, the invention relates to extracorporeal treatments which involve the removal of CO₂ and, at the same time, a hemofiltration treatment or a “dialysis for acute” treatment.

The aforementioned treatments, like any extracorporeal treatment, necessarily require access to the patient through a vein of appropriate size such as, for example, the Femoral vein, the Jugular vein, the Carotid vein or others at the discretion of the physician.

For a generic treatment of CO2 removal, blood is drawn from the vein by means of a catheter, often two-way. The blood is pumped by a pump into tubes that lead it to a medical device, called oxygenator, which provides the gaseous exchange by extracting the CO₂ and by administering O₂ by partial pressure difference through a gas-permeable membrane. After passing through the oxygenator the blood is returned to the patient, often through the same (two-way) catheter from which it was taken.

The circuit, i.e. the set consisting of catheter, tubes, active components (such as the oxygenator or hemofilters) is complemented by accessories such as pressure sensors, air bubble sensors, blood leak sensors, drippers, debuggers, accesses for blood samples and for the administration of drugs, anticoagulants, etc., using components known to specialists in the field. Such known components will be omitted in the description of the present invention and in the drawings for greater clarity and brevity, but the same components are to be considered present where necessary. In other words, these components or devices are not described or represented in the drawings but it is obvious that these devices are present because they are necessary for the operation of the apparatus.

It is known that the efficiency of gaseous exchange is proportional to the surface of the gas-permeable membrane and to the flow of blood passing through it, generally expressed in ml/min (milliliters per minute). The higher the flow, the more effective the extraction of CO₂.

It follows that, limited to the need to insert a catheter into the vein that is of the smallest size possible to obtain the desired effect, the goal of the aforementioned treatment, commonly called ECCO2R (Extra Corporeal CO2Remover), is to obtain the highest possible blood flow inside of the oxygenator.

In some devices, such as the one described in EP-1415673, the blood taken from the patient also passes through a component called “Hemofilter” which performs the functions of an artificial kidney.

Very often, in fact, simultaneously with the need to extract the excess of CO₂ present in the blood, there is the need to perform on the patient also a “dialysis for acute” treatment commonly referred to as Hemofiltration, CVVH (Continuous Veno-Venous Hemofiltration) or CRRT (Continuous Renal Replacement Therapy).

The hemofilter usually consists of a set of capillary tubes of appropriate material (e.g. Polysulfone or other) in which blood flows.

These capillary tubes are porous with a well-defined pore size. Through the aforementioned porosity of the capillary fibers the plasmatic water passes through, which can in this way be removed together with the waste substances from the same or conveyed in it, such as Urea and others (dialysis); the plasma water can also be mixed with a special solution (replacement liquid) slid out of the capillary fibers to reintegrate some useful substances (electrolytes) that would otherwise be dispersed in the extracted waste liquid. Said substances, by diffusion and/or by convection, pass through the same porosity of the capillary fibers reaching the blood flow in transit.

Hemofiltration treatment requires a maximum blood flow of around 200-250 ml/min; some hemofilters, although able to withstand higher flows, are designed for these values. Significantly higher flow rates may result in abnormal imbalances in dialysis and fluid exchange rates and also hemolysis problems.

There are medical studies and publications that demonstrate how the combination of these two treatments (extracorporeal CO₂ extraction and hemofiltration) provides a synergistic action to improve respiratory and renal failure, allowing the patient to increase the chances and speed of healing.

Some circuits place the oxygenator upstream of the hemofilter, others arrange it downstream. In any case, the flow of blood that passes through the two components is the same being the same arranged in series along the same blood line.

In the present description, specific reference will be made to the hemofilter as an active component acting in combination with the oxygenator in a veno-venous blood circuit.

In practice, the present invention is inserted in the specific technical field of the treatment of blood in which the blood is taken from a venous vessel of the patient, passes through an oxygenator and through a hemofilter, and is returned to the patient in a venous vessel. In other words, it is a veno-venous circuit in which only blood circulates.

Aim of the present invention is to optimize this dual extracorporeal treatment in a single blood treatment machine allowing, at the same blood flow rate withdrawn from (and subsequently returned) to the patient, to obtain a greater flow that passes through the oxygenator, contributing to the its better efficiency and duration, allowing to treat through the hemofilter a blood flow reduced and adequate to the specific hemofilter, generally designed to treat blood flows substantially lower than those suitable for the oxygenator.

It will therefore be possible to regulate the blood flow that will pass through the hemofilter to the values for which that particular hemofilter has been designed, including the significantly smaller pediatric hemofilters, and, at the same time, to send to the oxygenator a blood flow which is the sum of the flow coming from the patient and the flow passing through the hemofilter.

This particular characteristic is obtained with a circuit in a “ring” configuration where a main blood pump sucks the blood from the catheter inserted in a venous vessel of the patient and sends it to the oxygenator. A secondary blood pump (or other regulating means of the flow) draws a fraction of the main blood flow through a three-way connector disposed downstream of the oxygenator and, before it is returned to the patient, conveys it through the hemofilter.

The blood treated by the hemofilter and exiting the same is then conveyed, via another three-way connector, into the flow of blood that still has to transit into the oxygenator, upstream of it, and so on.

A “loop” is thus created, i.e. a secondary blood circulation ring independent of the main flow.

As an example, if the main blood pump sucks 500 ml/min from the patient's vessel and the secondary blood pump is set to a flow (optimal for a standard hemofilter) of 200 ml/min, after the three-way connector upwards disposed (also called first connector in the present description), in which the two flows add up, there will be 700 ml/min which will pass through the oxygenator, thus increasing its effectiveness thanks to this “virtual” flow increase.

In the circuits currently on the market, the flow of blood that passes through the oxygenator is equal to the flow of blood taken from the patient (in the above example of only 500 ml/min), while by using the present invention the flow is added to that passing through the hemofilter (therefore, 500+200, equal to 700 ml/min).

Subsequently, downstream of the oxygenator, the blood meets the downstream connector (also called second connector or second three-way connector in the present description), where a part of that flow (to be precise 200 ml/min referring to the previous example) is subtracted from the main flow and channeled into the “loop” to the hemofilter.

The remaining 500 ml/min will be returned to a patient venous vessel, purified by excess CO₂ and dialysis waste.

As an alternative and/or in addition to the two above-mentioned blood pumps, blood flow regulators may be provided which are able to exert a similar action on the blood.

This circuit offers numerous advantages, among which the ones described below can be mentioned.

It allows, at the same flow rate taken by the patient to submit a greater flow to the oxygenator's action (with a consequent higher efficiency of CO₂ extraction and lower risk of clots).

It allows, independently of the magnitude of the main blood flow, to accurately dose the blood flow to be passed through the hemofilter (without causing hemolytic damage and imbalance to the dialytic treatment).

It allows, in case of obstruction due to coagulation of the hemofilter (unfortunately frequent problem) to continue the treatment of CO₂ extraction simply blocking the secondary blood pump, obviously giving up the dialysis treatment allowing to continue the more expensive ECCO₂R treatment without damaging the relative equipment delegated to carry out this latter treatment.

If air bubbles are detected in the blood flow before returning to the patient, the bubbles can be automatically eliminated by stopping the main blood pump and circulating the blood with the bubbles through the hemofilter and the oxygenator where they will be eliminated. It allows, in case of temporary disconnection of the patient from the extracorporeal line (for example for the replacement of the catheter) to keep the blood circulating inside the oxygenator and the hemofilter thus avoiding the risk of clots, simply keeping the blood pump blocked main and running the secondary blood pump with recirculation function.

The ring conformation also avoids that the flow resistances of the two components are added together avoiding the frequent alarms of exceeding the pre-filter pressure which instead characterize the circuits which provide for the two components in cascade.

The objects and the advantages of the present invention will be more evident from the following description, which refers to the attached drawings which constitute an exemplary embodiment and in which:

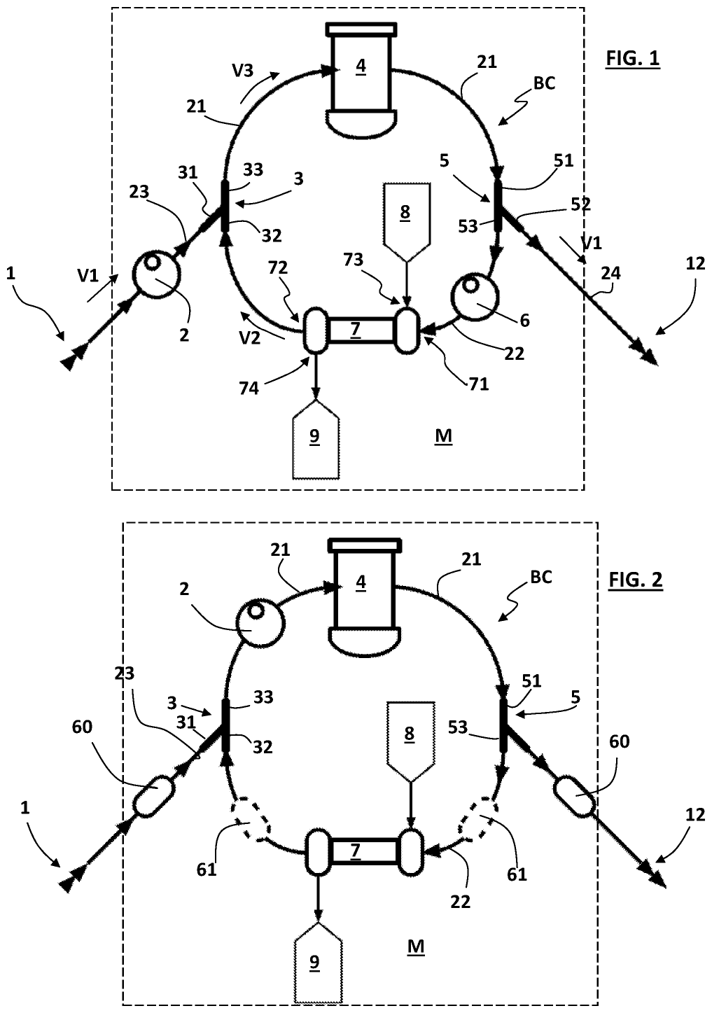

FIG. 1 illustrates schematically a possible embodiment of the invention;

FIG. 2 illustrates schematically another possible embodiment of the invention;

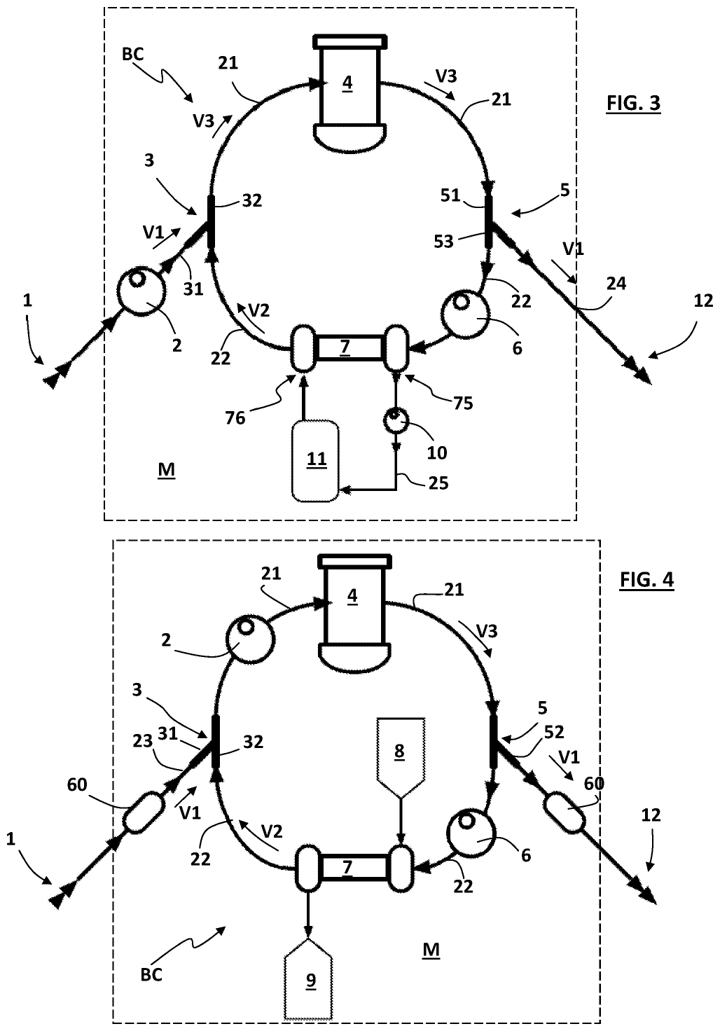

FIG. 3 illustrates schematically a further possible embodiment of the invention;

FIG. 4 illustrates schematically a further other possible embodiment of the invention.

With reference to the drawings of the attached figures, a blood treatment apparatus according to the present invention is of the type comprising a blood circuit (BC) defined by a main pump (2) and by one or more ducts through which the blood withdrawn from a patient passes to be treated at a given flow value (V1). The blood circuit is provided with an oxygenator device (4), which performs a treatment on the blood at a first flow value, and of a hemofilter (7), which performs a treatment on the blood at a second flow value, lower than the said first flow value.

The oxygenator (4) is arranged and acting on a first portion (21) of the blood circuit and the hemofilter (7) is arranged and acting on a second portion (22) of the blood circuit arranged in parallel with the first portion (21) so as to form a ring or “loop”.

The second portion (22) is connected to the first portion (21) downstream and upstream of the oxygenator (4), the blood entering the second device (7) being taken downstream of the oxygenator (4) and the blood in exit from the second device (7) being conveyed in the first portion (21) upstream of the oxygenator.

Flow regulation means (6; 60, 61), as described below, are provided for determining in the first portion (21) a flow (V3) of a value equal to the sum of the flow value (V2) of the second section (22) and the flow value (V1) of the blood taken from the patient and returned to the patient.

The blood circuit (BC) provided with the related devices (4) and (7) as well as the flow regulators (6, 60, 61) and the connecting pipes and connecting fittings, is entirely comprised by the apparatus object of the invention, schematically represented from a rectangle (M) in discontinuous line in the drawings. In practice, the apparatus in question is a single machine (M) with inside it oxygenator (4), hemofilter (7), pumps, ducts, etc.

According to the example of FIG. 1, said flow regulation means comprise an additional blood pump (6) arranged and acting on the second circuit portion (22). Furthermore, the downstream end of the second portion (22) or outlet end, through which the blood treated by the hemofilter (7) passes, is connected to the first portion (21) downstream of the main pump (2) and to upstream of said oxygenator (4).

The supplementary blood pump (6) can be placed upstream of the hemofilter (7).

Again with reference to the example of FIG. 1, the apparatus comprises a first “Y” connector (3) arranged downstream of the main pump (2) and a second “Y” connector (5) arranged downstream of the oxygenator (4). In particular, the first “Y” connector (3) comprises two input branches (31) and (32) and an output branch (33) and the second “Y” connector (5) comprises an input branch (51) and two output branches (52) and (53).

The input branches (31, 32) of the first connector (3) are connected, respectively, one (31) to the circuit portion downstream of the main blood pump (2) and the other (32) to the downstream end or of exit of the second portion (22). The output branch (33) is connected to the portion of circuit (21) on which the oxygenator (4) is arranged.

The input branch (51) of the second connector (5) is connected to the portion of circuit (21) on which the oxygenator (4) is arranged. The output branches (52, 53) are connected, respectively, one (52) to the outlet of the treated blood (portion 24) directed towards the patient's venous vessel, the other (53) to the upstream end or withdrawal end of the second portion (22).

According to the example of FIG. 2, said flow regulation means comprise a first regulator (60) arranged upstream of the main pump (2) and a second regulator (61) arranged on said second circuit portion (22). The second regulator (61), shown in a discontinuous line, can be arranged upstream or downstream of the hemofilter (7). The downstream end of said second portion (22), through which the blood treated by the hemofilter (7) passes, being connected to the first portion (21) upstream of the main pump (2) and upstream of said oxygenator (4) to exploit the suction power of said main pump (2).

In this embodiment, the apparatus comprises a first three-way connector (3) arranged upstream of the main pump (2) and a second three-way connector (5) arranged downstream of said oxygenator (4). The inlet and outlet branches of the three-way connectors are marked with the same references as the connectors in the example shown in FIG. 1.

The downstream end or output end of said second portion (22) is connected to the first connector (3), in particular to the input branch (32), the upstream end or withdrawal end of said second portion (22) being connected to the second connector (5), at the output branch (53).

In practice, the flow value (V2) in the second portion (22) is equal to said second flow value (i.e. the flow suitable for the correct functioning of the hemofilter), while the flow value (V3) in the first portion (21) is equal to the sum of the flow withdrawn (and returned) to the patient (also called first value V1) and of the flow (V2) circulating in the second portion (22).

According to the current practice and according to what is shown in the drawings of FIG. 1 and FIG. 2, a first tank (8) for a refill liquid is connected to the hemofilter (7). The so-called “dialysate” or waste liquid can be collected in a corresponding tank (9). Between the first tank (8) and the hemofilter (7), as well as between the same hemofilter (7) and the tank (9), pumps (not shown) may be provided, if necessary. In practice, the blood reaches the hemofilter at the inlet (71) to get out of it treated by the outlet (72). The conduits which connect the hemofilter (7) to the tanks (9, 8) are connected to the inlet (73) and to the outlet (74).

By way of example and in a manner obtainable with the example previously described and illustrated in FIG. 1, in FIG. 2 the main blood pump (2) sucks 700 ml/min, obtained overall from the sum of the blood flow taken to the patient from the point of access (1) equal to 500 ml/min (measured and regulated by the first flow regulator 60 downstream of the access point to the patient 1) and of the blood flow coming from the second portion (22), equal to 200 ml/min, through the three-way connector (3). As an alternative (or in addition, for safety reasons) to the first flow regulator (60) it will be possible to use the second flow regulator (61) positioned along the second portion (22) which will allow the passage of a flow of 200 ml/min (always as an example). The second regulator (61) can be placed upstream or downstream of the hemofilter (7).

Referring again to the illustrated examples, in particular in FIG. 1, the apparatus of the invention comprises a blood circuit which is suitably connected to venous vessels of the patient, for example by means of a catheter, to withdraw the blood to be treated. In the drawings it is marked with (1) the entry of the blood to be treated and with (12) the downstream outlet that brings the treated blood back to the patient. The two accesses (1) and (12) can be constituted by a double lumen catheter, for example of 13 Fr. For the measurement of the catheter the French (Fr) has been used, which is 3 times the diameter expressed in mm.

The blood is pumped downstream into the extracorporeal circuit by the main blood pump (2) in a section of tube (23) which defines the initial portion of the blood circuit of the apparatus.

The main blood flow from the initial portion of pipe (23) reaches the three-way connector (3) which connects the same portion of pipe (23) with the outlet end of the portion of pipe (22) on which it is arranged and acting the hemofilter (7) and the said first portion (21) of the circuit.

In the section of circuit (21) the blood flow (V3) is equal to the sum of the main flow (V1) produced by the pump (2) and the one (V2) coming from the hemofilter (7) produced by the additional pump (6).

The flow (V3) of the tube (21), sum of the two flows (V1) and (V2), enters the oxygenator (4). Downstream, from the oxygenator (4) through the second part of the portion (21) it reaches the three-way connector (5). At the connector (5) the blood flow (V2) for the hemofilter (7) is subtracted by the secondary pump (6) from the flow (V3).

In the terminal portion of the circuit (24) the original main blood flow (V1) remains, which is returned to the patient through access (12).

In the example of FIG. 2, a second flow regulator (60) is arranged and acting on the terminal portion of the circuit (24), similar to that on the initial portion of the circuit (23). Also in this case the blood flow that is returned to the patient through access (12) is the same as the original one (V1).

Again according to the example of FIG. 2, it is possible to eliminate the additional blood pump (6) by positioning the connector (3) upstream of the main blood pump (2), taking advantage of the vacuum generated by the latter.

This solution, apart from the advantage of not requiring a second blood pump, requires a “flow meter/regulator” (61) along the line (22) to check the correct distribution of the flows.

In the example of FIG. 3 a further embodiment of the invention is shown. In this embodiment, the structure and operating principle are similar to those of the previous examples, with the difference that the hemofilter (7) is provided with an outlet for the ultrafiltrate (75) and an inlet (76), connected to each other by an additional circuit (25) on which there is a pump (10) and a device (11) for the elimination of bicarbonates. In practice, while for the remaining part of the apparatus the operation is substantially similar to the previous examples, the additional circuit (25) is traversed by ultrafiltrate coming from the corresponding output (75) of the hemofilter (7). The ultrafiltrate is pumped by the ultrafiltered pump (10) which determines a flow of between 20 and 30% of the flow (V2) passing through the portion (22) of the circuit of the present apparatus. The passage through the device (11) allows the elimination of bicarbonates (bicarbonate ions), by means of physical, chemical, electrolysis or other means known and suitable for the purpose. The importance of eliminating bicarbonate ions lies in the fact that carbon dioxide is transformed into bicarbonate ions and the elimination of bicarbonate ions consequently increases the ability to eliminate CO₂ from the blood.

In the example of FIG. 4, the main pump (2) is arranged downstream of the connector (3) as in the example of FIG. 2. The blood circuit (BC) of the example of FIG. 4 is provided with a secondary pump (6) arranged as in the examples of FIGS. 1 and 3. In practice, the example of FIG. 4 has the main pump (2) arranged as in FIG. 2 but is not provided with the regulators (61) but with a secondary pump (6) and with possible regulators (60) at the portions (23) and (24) of the circuit.

In accordance with the present invention it is also possible to realize a process for the extracorporeal blood treatment which involves the passage of blood into a blood circuit defined by a main pump (2) and by one or more ducts through which the blood to be treated passes, the blood being withdrawn from a patient to a given flow value (V1); said circuit being provided with an oxygenating device (4), which performs a treatment on the blood at a first flow value, and of a hemofilter (7), which performs a treatment on the blood at a second flow value, lower than said first flow value.

Advantageously, the blood is passed through the oxygenator (4), which is arranged and acting on a first portion (21) of the blood circuit, and the hemofilter (7) which is arranged and acting on a second portion (22) of the blood circuit arranged in parallel with the first portion (21). The second portion (22) is connected to the first portion (21) downstream and upstream of the oxygenator (4), the blood entering the second device (7) being taken downstream of the oxygenator (4) and the blood in exit from the second device (7) being conveyed in the first portion (21) upstream of the oxygenator; there being provided means for regulating the flow (6; 60, 61) for determining in said first portion (21) a flow (V3) of a value equal to the sum of the flow value (V2) of the second portion (22) and of the value of the flow (V1) with which the blood is taken from the patient and returned to the same.

In other words, the process allows, at the same flow rate taken by the patient, to subject to the action of the oxygenator a greater flow (with consequent greater efficiency in extracting CO₂). It also allows, independently of the magnitude of the main blood flow, to accurately dose the blood flow to pass through the hemofilter (without causing hemolytic damage and imbalance to the dialysis). It allows, in case of obstruction due to coagulation of the hemofilter, to continue the CO₂ extraction treatment by simply blocking the secondary blood pump. If air bubbles are detected in the blood flow before returning to the patient, the bubbles can be automatically eliminated by stopping the main blood pump and circulating the blood with the bubbles through the hemofilter and the oxygenator where they will be eliminated. Another advantage of the process is that of allowing, in the case of temporary disconnection of the patient from the extracorporeal line (for example for the replacement of the catheter) to keep the blood circulating inside the oxygenator and of the hemofilter thus avoiding the risk of clots, simply keeping the main blood pump blocked and running the secondary blood pump with recirculation function.

In accordance with a further aspect of the process that can be implemented with the present invention, it is also possible to perform a treatment on the ultrafiltrate exiting the hemofilter (7). In practice, as previously described with reference to FIG. 3, the hemofilter (7) is provided with an output for the ultrafiltrate (75) and an inlet (76), connected to each other by an additional circuit. (25) on which there is a pump (10) and a device (11) for the elimination of bicarbonates. In practice, this additional step of the process involves making the ultrafiltrate pass through the additional circuit (25) with a flow of between 20 and 30% of the flow (V2) passing through the portion (22). The passage through the device (11) allows the elimination of bicarbonates (bicarbonate ions), by means of physical, chemical, electrolysis or other means known and suitable for the purpose.

The apparatus of the invention consists of a single machine (M) which allows the treatment of blood for the removal of CO₂ with maximum flows which normally do not exceed 500 ml/min with regard to the first flow value (V1) and 250 ml/min for the second flow value (V2). The access is carried out by veno-venous way in correspondence with peripheral vessels (for example femoral vein, subclavian vein or jugular vein) and preferably with a double-lumen catheter smaller than 15 Fr, for example of 13 Fr. In practice, the connections indicated by (1) and (12) in the drawings are both supported by the double lumen catheter. Naturally, the invention is not limited to what has been described and illustrated, but it can be widely varied with regard to the arrangement and the nature of the components used according to the inventive teaching described above and claimed below.

1. An apparatus for he extracorporeal treatment of blood with veno-venous access, of the type comprising a circuit (BC) inside the apparatus and defined by a main pump (2) and by one or more conduits through which the blood to be treated passes at a given flow value (V1), said circuit being provided with an oxygenator (4), which performs a treatment on the blood at a first flow value, and of a hemofilter (7), which performs a treatment on the blood at a second flow value, lower than said first flow value, apparatus characterized in that:
said oxygenator (4) is arranged and acting on a first portion (21) of the blood circuit and that the hemofilter (7) is arranged and acting on a second portion (22) of the blood circuit arranged parallel to the first portion (21);
said second portion (22) is connected to the first portion (21) downstream and upstream of the oxygenator (4), the blood entering the hemofilter (7) being taken downstream of the oxygenator (4) and the blood exiting the hemofilter (7) being conveyed in the first portion (21) upstream of the oxygenator; there being provided means for regulating the flow (6; 60, 61) for determining in said first portion (21) a flow (V3) of a value equal to the sum of the flow value (V2) of the second portion (22) and of the value of the flow (V1) with which the blood is taken from the patient and returned to the same;
said first portion (21) and said second portion (22) of the circuit are passed through only by blood.

2. Apparatus according to claim 1, characterized in that said flow control means comprise an additional blood pump (6) arranged and acting on said second circuit portion (22).

3. Apparatus according to claim 1, characterized in that said flow control means comprise an additional blood pump (6) arranged and acting on said second circuit portion (22), and in that the downstream end of said second portion (22), through which the blood treated by the hemofilter passes (7), is connected to the first portion (21) downstream of the main pump (2) and upstream. of the said oxygenator (4).

4. Apparatus according to claim 2, characterized in that said additional blood pump (6) is arranged upstream of the hemofilter (7).

5. Apparatus according to claim 1, characterized in that said flow regulation means comprise one or more flow regulators (60, 61) arranged upstream of said main pump (2) and on said second portion (22) of circuit, the end downstream of said second portion (22), through which the blood treated by the hemofilter (7) passes, being connected to the first portion (21) upstream of the main pump (2) and upstream of said oxygenator (4).

6. Apparatus according to claim 1, characterized in that the flow value (V2) in the second portion (22) is equal to said second flow value and that the flow value (V3) in the first portion (21) is equal to said first flow value, equal to the sum of said second flow value (V2) and of the flow value (V1) to which blood is withdrawn and returned to the patient.

7. Apparatus according to claim 2, characterized in that it comprises a first three-way connector (3) arranged downstream of said main pump (2), a second three-way connector (5) arranged downstream of said oxygenator (4), the downstream or outlet end of said second portion (22) being connected to said first three-way connector (3), the upstream or withdrawal end of said second portion (22) being connected to said second three-way connector (5).

8. Apparatus according to claim 5, characterized in that comprises a first three-way connector (3) arranged upstream of said main pump (2), a second three-way connector (5) arranged downstream of said oxygenator (4), the downstream or outlet end of said second portion (22) being connected to said first three-way connector (3), the upstream or withdrawal end of said second portion (22) being connected to said second three-way connector (5).

9. Apparatus according to claim 1, characterized in that the value of the flow (V2) in the second portion of the circuit (22) is substantially equal to 200 ml/min and the value of the flow (V1) to which the blood is drawn and returned to the patient is substantially equal to 500 ml/min.

10. Apparatus according to claim 1, characterized in that said hemofilter (7) is provided with an outlet for the ultrafiltrate (75) and an inlet (76), connected to each other by an additional circuit (25) on which there are a pump (10) and a device (11) for the elimination of bicarbonates.

11. Apparatus according to claim 1, characterized in that it is provided with connection means to a patient comprising a double-lumen catheter of dimensions smaller than 15 Fr.

12. Apparatus according to claim 1, characterized in that said circuit (BC) comprises said main pump (2), said oxygenator (4), said hemofilter (7), said flow regulation means (6; 60, 61) and the conduits through which the blood to be treated passes are contained in a single machine (M).

13. Apparatus according to claim 3, characterized in that said additional blood pump (6) is arranged upstream of the hemofilter (7).
